(19) United States
(12) Patent Application Publication
(10) Pub. No.: US 2009/0105779 A1
(43) Pub. Date: Apr. 23, 2009

(54) Lead Tracking Of Implantable Cardioverter-Defibrillator (ICD) And Cardiac Resynchronization Therapy (CRT) Devices

(75) Inventors: Thomas C. Moore, Livermore, CA (US); Mark Schneider, Williston, VT (US)

Correspondence Address: H. JAY SPIEGEL - H. JAY SPIEGEL & ASSOCIATES, P.O. BOX 11, MOUNT VERNON, VA, 22121, US

(73) Assignee: ASCENSION TECHNOLOGY CORPORATION, Burlington, VT (US)

(21) Appl. No.: 11/920,524

(22) PCT Filed: Mar. 6, 2007
(86) PCT No.: PCT/US2007/005676

§ 371 Date: Nov. 15, 2007

Related U.S. Application Data
(60) Provisional application No. 60/780,073, filed on Mar. 8, 2006.

(51) Int. Cl. A61N 1/365

(52) U.S. Cl. 607/20; 600/508; 607/24; 607/17

(57) ABSTRACT

Lead Tracking of Implantable Cardioverter-Defibrillator and Cardiac Resynchronization Therapy Devices improve upon the process of implantation of ICD-CRT devices, placing their leads, and improving the information fed back to the device and/or clinician. Tracking of the placement of the leads during implantation is accomplished along with monitoring the leads once implanted. Benefits include reducing the risk and complication rate, simplifying implantation procedure, and enabling the extraction of vital data not previously available. Leads are tracked to at least minimize the need to use fluoroscopy. Three dimensional tracking (10) is employed to facilitate obtaining of data that allows the surgeon to better visualize lead insertion and placement. Placement of the leads during a procedure requires use of an external tracking component along with means and method for tracking the implantable leads. Transmitting antennas (10, 110) are provided, equal in number to the number of degrees of freedom of tracking required. A link (50) between the sensor (70) and the computation unit (40) can be wired or wireless. Once leads are implanted, heart wall motion must be monitored via the tracking of the leads within a clinical or home environment. Such tracking of the leads may be accomplished in real time.

### BACKGROUND OF THE INVENTION

Heart failure occurs 550,000 times a year in the U.S., with an annual mortality of 266,000. Roughly 8% of people aged 65 or over have heart failure. There are presently 5,000,000 patients with heart failure in the U.S. and it is projected that by the year 2037 the number will double to 10,000,000. The annual cost of heart failure is $38 billion dollars, and 60% of the costs are related to hospitalization.

One of the treatments for people with moderate to severe heart failure is a device therapy known as cardiac resynchronization therapy (CRT). CRT can also be combined with implantable cardioverter-defibrillator (ICD) therapy to eliminate life-threatening tachyarrhythmias.

The ICD is an electronic device consisting of a generator and a lead system. The purposes of the device are to monitor heart rhythm and treat detected abnormal heart rhythms using variable modalities.

Improvements in generator technology have increased the options for treating tachyarrhythmias. These options now include electrical therapy (pacing), which is used to treat bradyarrhythmias. Thus, sustained ventricular tachycardia can be treated with competitive (overdrive) pacing or synchronized cardioversion, ventricular fibrillation can be treated with defibrillation, and bradycardia can be treated with pacing.

The lead system connects the generator of the ICD to the heart. This system allows heart rate to be detected and electrical therapies to be delivered. Lead technology has progressed rapidly in the past 10 years. Implantation no longer requires open-heart surgery and placement of electrical patches on the ventricle. Most ICDs now require only a single lead that can be placed transvenously. Since the generator forms one electrical pole of the cardioversion-defibrillation circuit, a second lead is not needed. However, devices that employ defibrillation patches on the ventricle are still in use, and these leads are usually retained when a generator is upgraded.

With permanent systems, endocardial leads are inserted into the venous system, usually via the subclavian, axillary, or cephalic vein, and advanced to the right ventricle and/or atrium. Newer pacing systems may have 2 atrial leads, one in the right atrial appendage and the other either in the coronary sinus or at the os of the coronary sinus, with the ventricular lead in the right ventricle, either at the apex or at the outflow tract. This dual site or biatrial pacing system is used to prevent or minimize bouts of atrial fibrillation. Another new pacing system is biventricular pacing with 2 ventricular leads, one in the right ventricle and the other in a venous branch of the coronary sinus.

Current ICDs store information about the arrhythmias. This information can be retrieved by interrogation of the ICD. This can be achieved by communicating via inductive coupling with an antenna placed over the device and attached to a programmer. The programmer is specific to the device of each manufacturer. Interrogation allows the physician to determine which electrical therapies have been given. Lead integrity and battery status are also checked. The device can then be adjusted to optimize detection and therapy parameters. Most ICDs also record the patient's electrocardiographic tracing at the time of arrhythmia detection. This information can be analyzed at follow-up visits to determine the nature of the arrhythmia and the efficacy of the electrical therapy that was given.

The variability of coronary venous anatomy sometimes makes the implantation of cardioverter defibrillator (ICD) and cardiac resynchronization therapy (CRT) devices difficult, even impossible to achieve. In addition to the placement of the electronic devices under the skin, single or multiple lead wires must be advanced venously under fluoroscopic guidance into one or more chambers of the heart muscle. Insertion is further hampered by the inability to inject contrast agents into veins and the 2D nature of fluoroscopic imaging.

In approximately 10% of cases, the procedure is aborted, typically due to the size, shape or location of the patient's vein. While the 10% may seem statistically acceptable, the percentage is problematic due to the high number of cases presented each year and the dire consequences of poor results. The ICD/CRT market is currently the largest cardiac device market with annual sales of approximately $10 billion worldwide.

In the normal heart, the heart's lower chambers (ventricles) pump at the same time and in sync with the heart's upper chambers (atria). When a patient has heart failure, often times the right and left ventricles do not pump together (dysynchrony). When the heart's contractions become out of sync, the walls of left ventricle (LV) do not contract at the same time. The heart has less time to fill with blood and is not able to pump enough blood out to the body. This eventually leads to an increase in heart failure symptoms.

Biventricular pacing keeps the right and left ventricles pumping synchronously together by sending small electrical impulses through the leads. When the atrium contracts, both ventricles are paced to contract at the same time, causing the walls of the left ventricle (the septal and free walls) to contract “in synch.” This allows the left ventricle (LV) and the right ventricle (RV) to pump together and also both walls of the left ventricle. Besides coordinating contractions, biventricular pacing reduces the amount of blood flow that leaks through the mitral valve and decreases the motion of the septal wall that separates the chambers of the heart. The end result is improved cardiac function.

Two leads are placed into a vein, and then guided to the right atrium and right ventricle of the patient's heart. The lead tips are attached to the heart muscle. The other ends of the leads are attached to the pulse generator, which is placed under the skin in the upper chest. The third, left ventricular lead is guided through the vein to a small vein on the back of the heart called the coronary sinus to pace the left ventricle.

It is interesting to note that the leads, once placed, are in an ideal position for measuring heart wall motions, if an appropriate mechanism could be ascertained. Quantitative measurement of left ventricular wall motion can improve clinical diagnosis by providing a more objective approach than qualitative analysis, which is subject to large inter-observer variability. It is known that wall motion analysis can successfully detect ischemia and provides an objective and quantitative approach for detecting and assessing the severity of disease. This information, besides being clinically important by itself, may further improve the control of heart rhythm management.

Many previous attempts at measuring heart wall motion utilize accelerometers, whose outputs are then integrated twice to determine displacement. Examples of this can be found in U.S. Pat. Nos. 5,480,412; 5,496,361; 5,628,777; 5,991,661; 6,002,963; 6,009,349 and 6,923,772. The drawbacks to this approach include (1) the fact that no absolute position reference is obtained, and (2) the inaccuracies that build up with a double integration of the data.

### SUMMARY OF THE INVENTION

The present invention relates to Lead Tracking of Implantable Cardioverter-Defibrillator and Cardiac Resynchronization Therapy Devices. The present invention improves upon the process of implantation of ICD-CRT devices, placing their leads, and improving the information fed back to the device and/or clinician. This is accomplished by tracking the placement of the leads during implantation and monitoring the leads once implanted. Benefits include reducing the risk and complication rate, simplifying the procedure, and enabling the extraction of vital data not previously available.

The present invention includes the following interrelated objects, aspects and features:
(1) In all of the embodiments of the present invention, leads are tracked so as to eliminate or at least minimize the need to use fluoroscopy. The present invention contemplates three dimensional tracking to facilitate obtaining of data that allows the surgeon to better visualize lead insertion and placement.
(2) In one embodiment of the present invention, a sensor having 5 degrees of freedom capability is employed, which consists of, for example, a coil of wire or a semi-conductor device. The sensor facilitates determination of position and orientation in 5 degrees of freedom. If desired, a sensor facilitating obtaining of 6 degrees of freedom data may be employed.
(3) Placement of the leads during a procedure requires use of an external tracking component along with means and method for tracking the implantable leads. Transmitting antennas are provided, equal in number to the number of degrees of freedom of tracking required. A link between the sensor and the computation unit can be wired or wireless.
(4) In another aspect, DC sensitive receiving sensors may be employed such as those using the Hall Effect or giant magnetostrictive devices. In either event, the number of devices is equal in number to the degrees of freedom of tracking required.
(5) Once leads are implanted, heart wall motion must be monitored via the tracking of the leads. This can be performed within a clinical or home environment. Such tracking of the leads may be accomplished in real time.
(6) In a further embodiment, permanent magnets employed in embodiments of the present invention may be replaced with electromagnets that also act as a dipole transmitter. Multiple electromagnets are time multiplexed to accommodate multiple leads.
(7) Other embodiments are also contemplated as will be described in greater detail hereinafter including those employing wireless sensors. Use of both an accelerometer and a tracking sensor is contemplated in which they are both located on an implanted lead to facilitate directly measuring heart chamber work function.
   Specific benefits of the present invention include:

1. Quantifiable assessment of cardiac performance over time.

2. Volumetric measurement within the beating heart.

3. Real-time 3D visualization of lead tips as they are advanced into the heart.

4. Ability to instantly visualize changes in lead placement caused by physical rotation of the proximal end of the insertion wire.

5. Simplification of lead placement for bi-ventricular tracing.

As explained hereinafter, one or more tracking means, such as static magnetic, pulsed DC magnetic, AC magnetic, and magnetic resonance can accomplish 3D localization of lead wires. A wireless tracking means is preferred to eliminate fragile wiring and increase reliability. 5 degrees-of freedom (5DOF) tracking is the preferred method for all position and orientation methods since this requires the simplest sensing means and design. 5DOF tracking requires the minimum number of devices, one per lead/device, and provides 3 Cartesian coordinates (x,y,z) and two orientation parameters.

Accordingly, it is a first object of the present invention to provide lead tracking of implantable cardioverter-defibrillator and cardiac resynchronization therapy devices.

It is a further object of the present invention to provide such a method in which the process of installation of implanted devices on the heart is monitored in 5 degrees of freedom.

It is a still further object of the present invention to provide such a method in which the process of installation of implanted devices on the heart is monitored in 6 degrees of freedom.

It is a still further object of the present invention to provide such a method in which tracking is accomplished through wired connection between a sensor and monitoring device.

It is a yet further object of the present invention to provide such tracking using wireless technology.

It is a still further object of the present invention to facilitate monitoring of heart wall motion via tracking of leads.

These and other objects, aspects and features of the present invention will be better understood from the following detailed description of the preferred embodiments when read in conjunction with the appended drawing figures.

### BRIEF DESCRIPTION OF THE DRAWINGS

FIG. 1 shows a schematic representation of a system in which tracking of lead placement is accomplished by a wired or wireless link.

FIG. 2 shows a further schematic representation of a system in which tracking of lead placement is accomplished by a wired or wireless link.

FIG. 3 shows a schematic representation of a system including a wireless sensor assembly.

FIG. 4 shows a further schematic representation of a system in which tracking of lead placement is accomplished by a wired or wireless link.

FIG. 5 shows an embodiment employing permanent magnets and electromagnets.

FIG. 6 shows a further schematic representation of a system in which tracking of lead placement is accomplished by a magnet or an electromagnet.

FIG. 7 shows a schematic representation of a sheath 380 employed in lead placement.

FIG. 8 shows a schematic representation of a heart with leads implanted thereon.

### SPECIFIC DESCRIPTION OF THE PREFERRED EMBODIMENTS

All embodiments disclosed herein allow the leads to be tracked, eliminating, or at least minimizing, the use of fluoroscopy. This additional 3-dimensional tracking data also allows the surgeon to better visualize lead insertion and placement, improving the outcome of the procedure. This is accomplished by any of the techniques known in the prior art, in which 3D tracking data is fused with pre-acquired, or real time imaging data from 2D or 3D sources such as MRI, CAT and PET scans.

One preferred embodiment uses a 5 degrees-of-freedom (5DOF) sensor, typically a coil of wire or a single semiconductor device. Such sensors are capable of determining position in 3 dimensions (e.g., x,y,z Cartesian coordinates) and two device orientation parameters such as two of pitch, roll and yaw. A 5DOF tracking system typically uses N transmitters (field generators) and M sensors, with N≧5 and M=1. MN must be ≧5 and is typically 9 for best tracking results. The N measurements are typically used in a least squares algorithm to determine position and orientation. Examples of these types of systems can be found in U.S. Pat. Nos. 4,622,644; 4,710,708; 5,592,939; 6,052,547; 6,226,547; 6,385,482; 6,427,079; 6,484,118; 6,690,963; 6,701,179 and 6,836,745, incorporated by reference. Of course, 6DOF tracking is also feasible using techniques known in the art. Wireless variations are also available. All of the methods provide accurate position and/or orientation measurement capability.

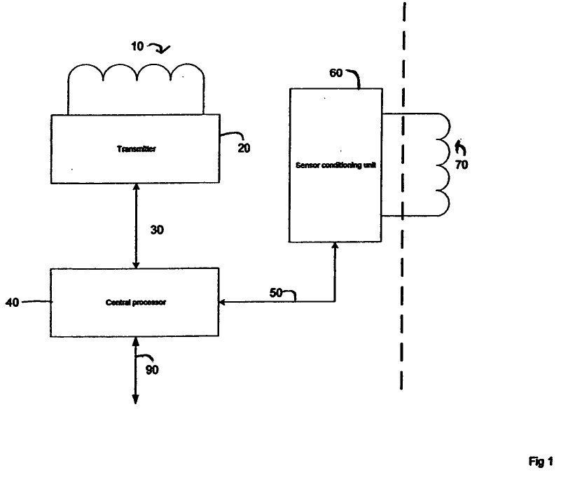

Safely and accurately placing the leads during a procedure requires using an external tracking component (versus an embedded one) along with a means and method of tracking the implantable leads. The general configuration for tracking lead placement is schematically shown in FIG. 1. A set of transmitting antennae 10 are provided, the number of which depends on the degrees-of-freedom (DOF) of tracking required. Driver 20 provides the excitation to the antennae 10. Antennae 10 and driver 20 may be RF based. The computation unit 40 may control the excitation over link 30. Computation unit 40 calculates the position and orientation of sensor(s) 70. Link 30 can be a physical link such as wire or fiber optics or could be a wireless link (not shown in FIG. 1). The excitation is measured or sensed by sensor(s) 70. The sensed signals are further processed, as required by conditioning unit 60. Conditioning unit 60 can include analog and digital conditioning, as well as signal processing provided by a digital process. A link 50 connects computation unit 40 to conditioning unit 60 to exchange conditioned, sensed data via appropriate means. Link 50 can be a physical link such as wire or fiber optics or could be a wireless link (not shown). A link 90 connects computation unit 40 to the user. Link 90 can be a physical link such as wire or fiber optics or could be a wireless link. Link 90 may also be part of the ICD's electro-magnetically coupled communication link. The computation unit provides the position and orientation of the sensor 70 with respect to the transmitting antennae 10. Any one of these devices can be repeated, multiplexed or duplicated in any manner known in the art to achieve tracking of more than one sensor.

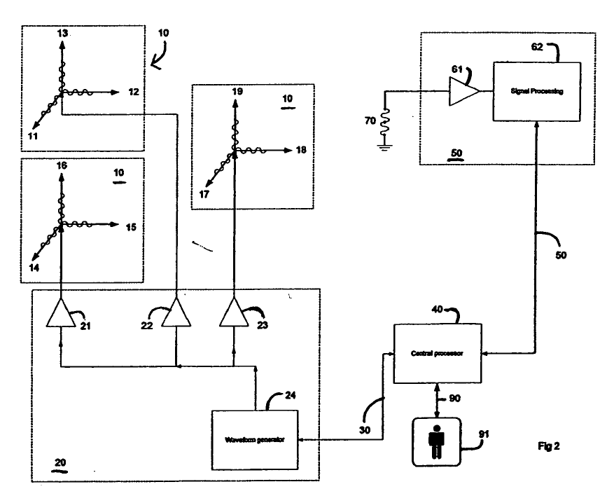

FIG. 2 is representative of most of the hardware found in 5DOF and 6DOF electromagnetic tracking systems and expands the depiction of FIG. 1. In the configuration shown in FIG. 2, transmitter arrays 10 consist of 3 sets of orthogonal coils. In other configurations, the coils comprising 10 are distributed at known positions and orientations, not necessarily orthogonal, and may be planar. As noted above, more or fewer transmitter coils may be used. Amplifiers 21-23 are shown as being time multiplexed between the antennae comprising arrays 10. They may be current or voltage sources, as required. In other configurations, there can be one amplifier per transmitter coil (11-19), or a single amplifier may be multiplexed in time, as is known in the art. Depending on the amplifier and tracker configuration, waveform generator 24 can generate pulsed DC, pulsed AC, ramps, pseudorandom noise, multi-frequency sinusoids, etc. These waveforms are amplified by amplifiers 21-23 and cause the magnetic fields at antennae 11-19 to be generated. Waveform generator 24 can be digitally controlled from central processor 40 or can have fixed sequences of waveforms programmed into it. The fields generated by the antennae are sensed by sensor 70. If sensor 70 is a coil, then the induced voltage is the time derivative of the current flowing in antennae 11-19). In other configurations, sensor 70 could be a semiconductor device such as a Hall-Effect sensor, a GMR sensor, a magnetometer or other device known in the art. Typically, signals generated across the sensor are amplified at amplifier 61, which may have adjustable gain. This gain can be adjusted by circuitry associated with the amplifier 61, the signal processor 62 or the central processor 40, depending on the sensor and the tracker architecture. Further, sensor 70 and sensor processing unit 60 could be configured to act as a variable oscillator, whose frequency is controlled by the impinging electromagnetic fields from antennae 11-19. An example of this type of sensor is disclosed in U.S. Pat. No. 6,84,406 (?), the disclosure of which is incorporated herein by reference. Signal processor 62 can perform many other functions and can contain both analog and digital components. Multiplexing, filtering, synchronous demodulation, integration, FFT, correlation, and A/D conversion, among other functions, are carried out in this section. The type of transmitter excitation and the sensor means determines what functions are performed. These functions may or may not be under central processor 40 control. The output of signal processor 62, typically a digital signal representing the sensed field, is further processed in an algorithm as explained below to determine the position and orientation of the sensor 70. The position and orientation is communicated to the user 91 via an appropriate interface under central processor 40 control. Of course, sensor and transmitter operation can be reversed, with multiple sensors and a single 5DOF transmitter. This is disclosed in U.S. Pat. No. 5,211,165.

U.S. Pat. No. 6,836,745 generally discusses the algorithmic method of position and orientation solution for these types of trackers. Bladen, in U.S. Pat. No. 6,757,557 discloses both 5DOF and 6DOF solutions to this type of tracker. Differences in antennae geometry sometimes yield different methods of solution. Examples of this are disclosed in U.S. Pat. Nos. 5,592,939; 6,427,079 and 6,701,179, among others. When antennae 11-19 are planar, computational algorithms such as those in U.S. Pat. Nos. 5,752,513; 6,052,610; 6,226,547 and 6,690,963 are possible. Some algorithms work for many different transmitter and sensor configurations and are disclosed in U.S. Pat. Nos. 4,622,644; 4,710,708; 6,073,043; and 6,427,079, among others.

Tracking a coil based sensor location based on the fields generated by an MRI is disclosed in U.S. Pat. Nos. 5,307,808; 5,353,795; 5,947,900; 6,289,233; and 6,687,530, also incorporated by reference. This system uses the pulsed gradient fields developed by the MRI as the “transmitters.” The sensed sensor signal yields a signal that, after processing, is the position of the sensor coil.

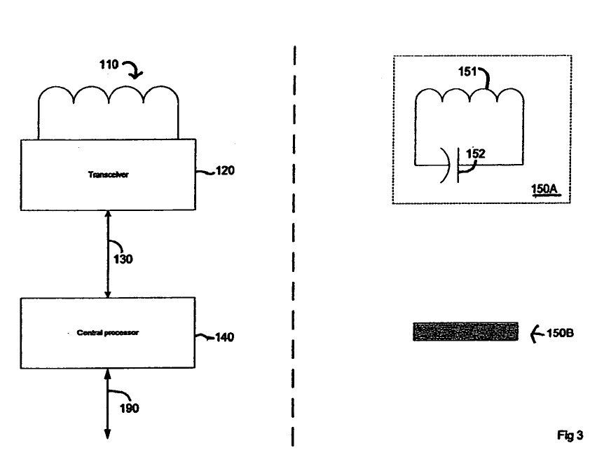

In another preferred embodiment, with reference to FIG. 3, a sensor 150 that is attached to a lead is wireless. In one such embodiment, such a sensor 150A is comprised of a small coil of wire 151 with a capacitor 152 across it. This forms a tuned circuit that resonates at a frequency determined by the coil inductance and the capacitance of the capacitor. At high enough frequencies, a coil of wire will act like a resonant circuit as parasitic elements become more pronounced. This embodiment relies on generating a known magnetic field, causing the coil to resonate, the coil's resonance being detectable via another sensor. This resonance-detecting sensor 110 can also be the original generator of the magnetic field, used in a multiplexed mode. The detected value can be used to determine position and/or orientation of the wireless sensor coil. Examples of such systems are disclosed in U.S. Pat. Nos. 4,642,786; 5,727,552; 6,026,818; and 6,997,504, all incorporated herein by reference.

In FIG. 3, region A, the wireless sensor assembly 150A, comprised of coil 151 and capacitor 152, forms a wireless method of providing lead tracking. Reference numeral 110 refers to a set of transmitting and receiving antennae, the number of which depends on the degrees-of-freedom (DOF) of tracking required. Driver/transceiver 120 provides the excitation to the antennae 110 and is also used to receive retransmission from sensor assembly 150A. Antennae 110 and driver/transceiver 120 may be RF based. The central processor 140 may control the excitation and reception over link 130. Central processor 140 calculates the position and orientation of sensor(s) 150A. Link 130 can be a physical link such as wire or fiber optics or could be a wireless link. The excitation of antennae 110 causes the sensor assembly 150A to resonate. The resonating assembly generates its own magnetic field, which is sensed by the receiving portion of antennae 110 and driver/transceiver 120. The received signals are further processed in the driver/transceiver 120 in a manner similar to conditioning unit 60 (FIG. 1). Link 130 connects central processor 140 to transceiver 120. Link 130 can be a physical link such as wire or fiber optics or could be a wireless link. A link 190 connects computation unit 140 to the user. Link 190 can be a physical link such as wire or fiber optics or could be a wireless link. Link 190 may also be part of the ICDs electro-magnetically coupled communication link. The computation unit provides the position and orientation of the sensor with respect to the transmitting antennae. Driver/transceiver 120 and antennae 110 can be separated into separate transmission and reception means to facilitate their use in a procedure.

In an alternative embodiment depicted in the region B in FIG. 2, the wireless sensor attached to the lead is made of a material that physically modifies a generated magnetic field. Such materials can be highly permeable ones, like mu-metal. This embodiment relies on generating a known magnetic field, and correlating the change in magnetic field due to the proximity of the permeable material with another sensor. Without the permeable material, the other sensor detects a specific field reading. The variation of that field reading due to the introduction of the permeable material can be used to determine position and/or orientation of the permeable material. Examples of such systems are disclosed in U.S. Pat. No. 6,076,007; WO96/31790, and U.S. Published Patent Application No. 2004/0254453, incorporated herein by reference. This embodiment is illustrated in FIG. 3, region B, where sensor assembly 150A is replaced with permeable material 150B. Permeable material 150B acts similarly to the sensor assembly 150A to “retransmit” or materially affect the externally generated field from transceiver 120 and antennae 110 back to antennae 100 and transceiver 120.

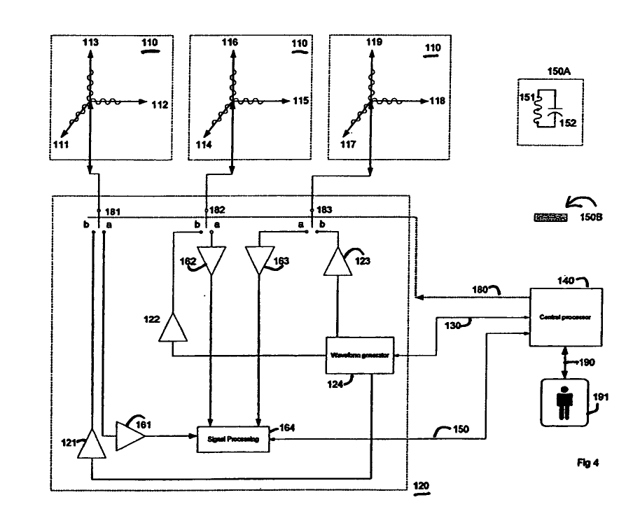

Further details on the above “retransmission” type system are detailed in FIG. 4. In the configuration shown, arrays 110 consist of 3 sets of orthogonal coils. In other configurations, the coils comprising 110 are distributed at known positions and orientations, not necessarily orthogonal, and may be planar. As noted previously, more or fewer coils may be used. The coils act as both transmitters and receivers. During operation, the antennae arrays 110 are time multiplexed between amplifiers 161-163 and amplifiers 121-123 via multiplexing switches 181-183, which ate controlled by central processor 140 via control line 180. Position a of the multiplexing switches places the antennae 110 in sensing mode, while position b places them in field generating mode. Amplifiers 121-123 may be current or voltage sources, as required. In other configurations, there can be one amplifier per transmitter coil (111-119), or a single amplifier may be multiplexed in time, as is known in the art. In further configurations, there can be one amplifier per sensor coil (111-119), or single amplifiers (161-163) may be multiplexed in time, as is known in the art. Depending on the amplifier and tracker configuration, waveform generator 124 can generate pulsed DC, pulsed AC, ramps, pseudorandom noise, multi-frequency sinusoids, etc., but preferentially generates short pulses. These waveforms are amplified by amplifiers 121-123 and cause the magnetic fields at antennae 111-119 to be generated. Waveform generator 124 can be digitally controlled from central processor 140 or can have fixed sequences of waveforms programmed into it. The fields generated by the antennae cause sensor 150A to resonate at its resonant frequency. As is known in the art, an L-C circuit (formed from coil 151 and capacitor 152) will resonate at its resonant frequency when excited by an impulsive function, such as that generated by antennae 111-119. When sensor 150A resonates, it generates a magnetic field discernible from the excitation. Antennae 111-119 sense this excitation. This is caused to occur when multiplexing switches 181-183 are at position a.

Signals generated across the sensors are amplified at amplifier 161-163, which may have adjustable gain. This gain could be adjusted by circuitry associated with the amplifiers, the signal processor 164 or the central processor 140, depending on the sensor and the tracker architecture. In another configuration, sensor 150B could be a material that affects the generated fields from antennae 111-119 in a manner similar to a resonant circuit. Such a material might be mu-metal, or other high permeability material. Signal processor 140 can perform many other functions and can contain both analog and digital components. Multiplexing, filtering, synchronous demodulation, integration, FFT, correlation and A/D conversion, among other functions, are carried out in this section. The type of transmitter excitation and the sensor means determines what functions are performed. These functions may or may not be under central processor 140 control. The output of signal processor 164, typically a digital signal representing the sensed field, is further processed in an algorithm to determine the position and orientation of the sensor 150. The position and orientation is communicated to the user 191 via an appropriate interface under central processor 140 control. As is also known in the art, antennae 111-119 could be physically split into two separate arrays, 111a-119a for field generation, and 111b-119b for sensing. The operations description would not change. While this introduces additional hardware, it may be advantageous to do so for particular medical procedures where placement, size, etc. add additional design constraints. Algorithms for this type of tracker are the same as for the ones described for FIG. 1 and FIG. 2.

FIGS. 3 and 4 also exemplify the tracking system disclosed in U.S. Pat. Nos. 6,812,842; 6,822,570; 6,838,990; 6,977,054; 7,026,927 and 7,176,798, all incorporated herein by reference. These disclosed systems all use a resonant tracking device, a pulsed method of excitation and a sensor array for receiving the resonant sensor response. Implementation details and signal processing means are described, but the basic configuration remains the same.

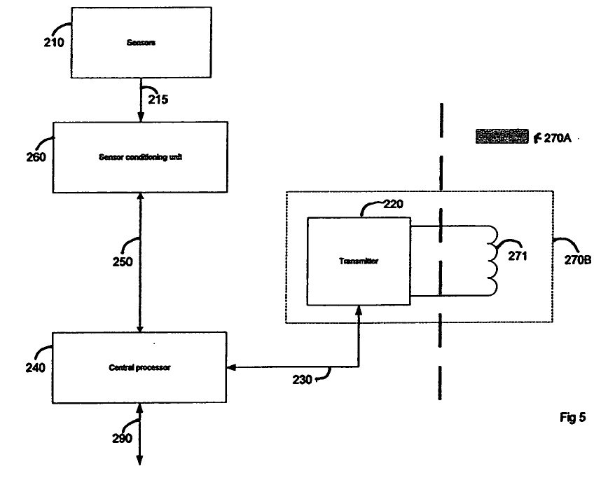

In another embodiment (see FIG. 5), a set of DC sensitive receiving sensors 210 are shown schematically, such as Hall effect or giant magnetostrictive devices, the number of which depends on the degrees-of-freedom (DOF) of tracking required. These antennae are situated to provide 5DOF tracking of a magnet 270A, which is situated at the distal end of the lead or in the sheath (not shown). The magnetic field from magnet 270A is measured or sensed by sensor(s) 210. The sensed signals are further processed, as required, by conditioning unit 260 connected to sensors by link 215. Conditioning unit 260 can include both analog and digital conditioning, as well as signal processing provided by a digital process. A link 230 connects central processor 240 to conditioning unit 260 to exchange conditioned, sensed data via appropriate means. Central processor 240 calculates the position and orientation of magnet(s) 270A. Link 230 can be a physical link such as wire or fiber optics or could be a wireless link. The computation unit provides the position and orientation of the magnet with respect to the receiving antennae. A link 290 connects central processor 240 to the user. Link 290 can be a physical link such as wire or fiber optics or could be a wireless link. Link 290 may also be part of the ICD's electro-magnetically coupled communication link. Any one of these devices can be repeated, multiplexed or duplicated in any manner known in the art to achieve tracking of more than one sensor. Sensors 210 can be a combination of vector or gradient sensitive devices, or could be arranged to indirectly provide said information.

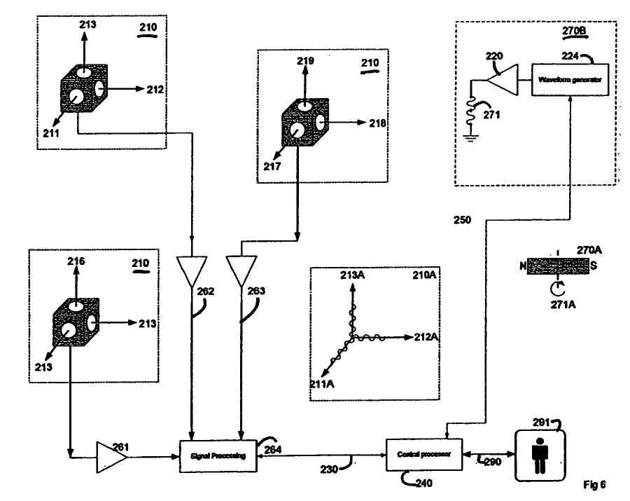

Further details of the above can be found in FIG. 6. In the configuration shown, arrays 210 consist of 3 sets of orthogonal sensing devices (211-219). In other configurations, the sensing devices comprising 210 are distributed at known positions and orientations, not necessarily orthogonal, and may be planar. These devices can be a set of DC sensitive receiving sensors, such as Hall effect or giant magnetostrictive devices if the excitation is strictly DC, for example, from a permanent magnet 270A. If the permanent magnet can be rotated about axis 271A, sensing devices 211-219 need only be sensitive to changes in the magnetic field. These could then be coils, for example, as exemplified in 210A, comprised of components 211A-213A, etc. Magnet 270A could also be replaced by assembly 270B, comprised of transmitting coil 271, amplifier 220 and waveform generator 224. These devices have been described previously under the description of 11, 21 and 24, respectively.

As noted previously, more or fewer sensing devices may be used. During operation, the antennae arrays 210 are time multiplexed between amplifiers 261-263. In further configurations, there can be one amplifier per sensor device (211-219), or single amplifiers (261-263) may be multiplexed in time, as is known in the art. Magnet 270A generates a magnetic field. Antennae 211-219 sense this excitation. Signals generated across the sensors are amplified at amplifier 261-263, which may have adjustable gain. This gain can be adjusted by circuitry associated with the amplifiers, the signal processor 264 or the central processor 240, depending on the sensor and the tracker architecture. Signal processor 264 can perform many other functions and can contain both analog and digital components. Multiplexing, filtering, synchronous demodulation, integration, FFT, correlation and A/D conversion, among other functions, are carried out in this section. The type of transmitter excitation and the sensor means determines what functions are performed. These functions may or may not be under central processor 240 control. The output of signal processor 264, typically a digital signal representing the sensed field, is further processed in an algorithm to determine the position and orientation of the sensor 270A or 270B. The position and orientation is communicated to the user 291 via an appropriate interface under central processor 240 control. Examples and algorithms for this type of tracker can be found in U.S. Pat. Nos. 4,622,644 and 6,052,610.

If the sensing devices of FIG. 6 are gradiometers, and there are a sufficient number of them, position and orientation can also be determined. U.S. Pat. No. 6,385,482 discloses such a system. Algorithms for this type of system can also be found in “Dipole Tracking with a Gradiometer,” W. M. Wynn, Naval Ship Research and Development Center, Informal Report NSRDL/PC 3493, January 1972.

The magnetic field generated by an external transmitter, such as 10 (FIG. 1) or 110 (FIG. 3), can be used to supply power to the ICD-CRT device. This power can be used to recharge the battery, if the ICD-CRT is so designed to accomplish this, or could be used to power just the tracking components that have been added to the ICD-CRT. This would occur inductively, and is well known in the art.

Once leads are implanted, the important process becomes monitoring of the heart wall motion via the tracking of the leads. This can be performed using any of the embodiments above within a clinical or home environment. In embodiments disclosed in FIGS. 1, 3 and 5, various components on the left side of the dashed line within the figures can be included in the ICD-CRT. Depending on space constraints, just the transmitter or receiver could be incorporated with position and orientation data calculated outside the body, or the receiver and the computational unit calculating and logging the position and orientation data within the ICD.

A simple method of measuring heart wall motion, based on an implantable magnet and Hall Effect sensors, is disclosed in U.S. Pat. No. 5,161,540, and incorporated herein. This method only provides a range measurement between the Hall elements and the magnet. The preferred approach is to use more advanced, active magnetic tracking technology. Many variations of this technology are available.

It is also advantageous to provide tracking of the leads, and hence the heart wall motion, in real time. This information can then be used with the ICD-CRT unit to further enhance and adapt the therapy applied by this device. In embodiments disclosed in FIGS. 1, 3 and 4, various components on the left side of the dashed line within the figures can be included in the ICD-CRT depending on size constraints, type of transmitter excitation and ICD-CRT construction.

In another alternative embodiment, with reference back to FIG. 5, the magnet 270A is replaced by an electromagnet 271. This then acts as a dipole transmitter. Multiple electromagnets are then time multiplexed to accommodate multiple leads. Depending on the excitation generated by transmitter 220, sensors 210 can be a set of DC sensitive receiving sensors, such as-Hall Effect or giant magnetostrictive devices if the excitation is strictly DC. For pulsed DC or AC transmitter excitation, sensors 210 need only be sensitive to changes in the magnetic field. These could then be coils, for example. These antennae are situated to provide 5DOF tracking of an electromagnet 271, which is situated at the distal end of the lead or in the sheath. The magnetic field from 271 is measured or sensed by sensor(s) 210. The sensed signals are further processed, as required, by conditioning unit 260. Conditioning unit 260 can include both analog ad digital conditioning, as well as signal processing provided by a digital process. A link 230 connects central processor 240 to conditioning unit 260 to exchange conditioned, sensed data via appropriate means. A link 250 connects central processor 240 to transmitter 220 to control the transmitter excitation via appropriate means. Central processor 240 calculates the position and orientation of magnet(s) 271. Links 230 and 250 can be physical links such as wire or fiber optics or could be wireless links. The central processor provides the position and orientation of the magnet with respect to the receiving antennae. A link 290 connects central processor 240 to the user. Link 290 can be a physical link such as wire or fiber optics or could be a wireless link. Link 290 may also be part of the ICD's electro-magnetically coupled communication link. Any one of these devices can be repeated, multiplexed or duplicated in any manner known in the art to achieve tracking of more than one sensor. Sensors 210 can be a combination of vector or gradient sensitive devices, or could be arranged to indirectly provide said information. The number of sensors is dependent on the degrees-of-freedom (DOF) of tracking required. FIG. 6 provides further details.

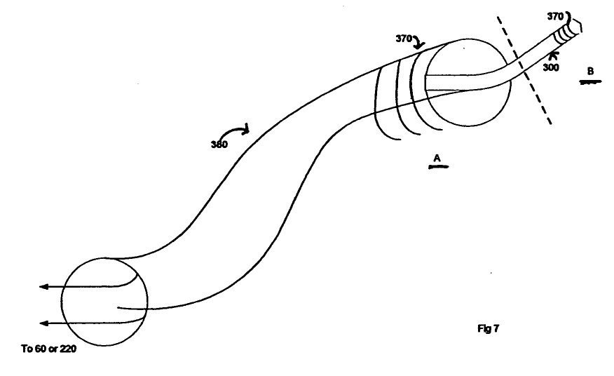

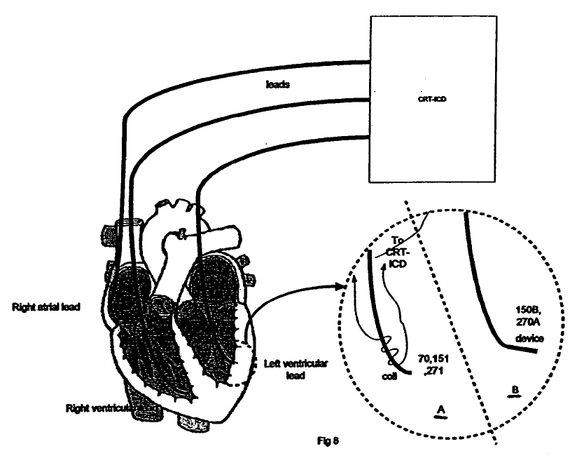

These devices 70, 150A, 150B, 270A, 270B and 271 are associated with the lead placement mechanism and can either be at the end of the sheath or at the end of the lead (see FIG. 7). FIG. 7, region A, shows a 5DOF coil 370 at the tip of sheath 380. It is understood that any of the devices 70, 150A, 150B, 270A, 270B and 271 could be placed at the tip of the sheath. During a lead implantation, the sheath is used to guide the lead to its attachment point in the heart. Likewise, FIG. 7, region B, shows a 5DOF coil at the tip of lead 300. It is understood that any of the devices 70, 150A, 150B, 270A, 270B and 271 could be placed at the tip of the lead. In the case of the sheath, the device can be embedded in the sheath at manufacture, or could be placed there during the procedure. In the case of the lead, the device is preferably manufactured with the lead, although it may be possible to attach the device at the start of or during the procedure. In wired device embodiments, the device leads are attached to conditioning unit 60 (FIG. 1) or 220 (FIG. 5). It may even be advantageous to track both sheath and lead at the same time. FIG. 8 shows the various lead tracking devices attached to leads. Leads are implanted in the heart and attached to the ICD-CRT.

In still another embodiment, the wireless sensor attached to the lead is any material (including a coil of wire) that could cause a highly sensitive, null-balanced detector to become unbalanced (like a metal detector). The amount of unbalance can be correlated to the position and/or orientation of the sensor. Examples of such systems are disclosed in U.S. Pat. Nos. 6,418,335 and 6,541,966, all incorporated herein by reference.

In another embodiment, a sensor on a lead measures magnetic fields from a field generator and a corresponding signal is then transmitted using power from an attached source or from the ICD itself. Its signal is then detected by a sensor and correlated to the position and/or orientation of the transmitter. Examples of such systems are disclosed in U.S. Pat. Nos. 5,443,066; 6,995,729; 6,233,476; and U.S. Published Patent Application No. 2005/0099290, incorporated herein by reference.

In still another embodiment, the wireless sensor attached to the lead sends its sensed field measurements to the ICD device, which stores it for downloading to the clinician at a later time.

The device/lead combinations can be secured entirely within the heart wall or LV using corkscrew, helical anchor, harpoon, threaded member, hook, barb, fastener, suture, mesh or coating for receiving fibrous tissue growth.

Once at least one lead is placed, the sensor on the lead now functions to enable real time tracking of the absolute heart wall motion. The heart wall displacement is directly correlated to the maximum and minimum volume of heart chamber containing the sensor. This changing volume is useful for determining overall heart efficiency (assuming normal valve operation) and pumping capacity. When more then one lead with accompanying sensors are in place, the sensors on the leads now function to enable the “real time” tracking of multi-chamber heart wall motion. The relative motion of the multiple sensors will correspondingly be related to a direct measure of the mechanical efficiency of the heart (assuming normal valve operation). The displacements correspond to known hemodynamic indicators, such as volumetric measurement, and are shown to be strongly suggestive of hemodynamic performance. Tracking also provides information regarding how well synchronized the chambers are. This information could then be used to adjust the ICD to deliver better treatment. This could be done in a real-time mode, if the ICD has enough internal logic, or via the clinician.

When both an accelerometer and a tracking sensor are located on an implanted lead then the heart chambers' work function can be directly measured. Work occurs when a force is exerted over a displacement. Assuming a constant and determinable heart mass, the accelerometer measures the changing force (mass times acceleration) over time, where the tracking sensor will measure the absolute displacement over time. Thereby, the real-time work function (force times displacement) of the heart chamber is measured. This information could then be used to adjust the ICD to deliver better treatment. This could be done in a real-time mode, if the ICD has enough internal logic, or via the clinician. The work function may be utilized to control the ICD to optimize the heart muscle performance thereby optimizing or enhancing reverse heart remodeling. Of course, differentiating the sensor data one or more times can also provide velocity and acceleration information.

Another application that would not require tracking of leads occurs during an open-heart surgery. Wireless sensors can be attached to the heart walls during an open-heart procedure and can still be used for monitoring heart wall motion. This does not require any active pacing or defibrillating devices.

Another application is to provide a method for stabilizing heart motion when used with image fusion technologies. Knowing the motion of the heart enables the heart to be mathematically stabilized when overlaid on a fixed graphic image of the heart. This is valuable when used with pre-acquired images.

In certain embodiments, or when one is dealing with older pacemakers, a single lead is all that is necessary to provide heart motion wall feedback. The relative motion of the heart wall over time still provides valuable information to the clinician regarding the state and rate of heart failure.

The low cost magnetic generator/tracking can also be rapidly adapted for deploying into the home cardiac monitoring market. A home monitoring solution would provide continuous sleep time monitoring of “real time” cardiac mechanical performance. Further, the magnetic tracking field can be of sufficient field strength to provide power to the ICD/CRT implantable device. This is used to offset the power consumption required by the additional processing and communication requirements of the tracking system, and thereby maintaining and/or extending the device's battery life.

Transmission and reception means may be reversed as is known in the art. Depending on procedure and application, two different means may be required for tracking, e.g., an internal tracking method once the leads are placed, and an external tracking method for placing the leads. As such, an invention has been disclosed in terms of preferred embodiments thereof, which fulfill each and every one of the objects of the invention as disclosed, and provide new and useful lead tracking of implantable cardioverter-defibrillator (ICD) and cardiac resynchronization therapy (CRT) devices of great novelty and utility.

Of course, various changes, modifications and alterations in the teachings of the present invention may be contemplated by those of ordinary skill in the art without departing from the intended spirit and scope thereof.

As such, it is intended that the present invention only be limited by the terms of the appended claims.

1. A system for tracking absolute position and orientation of an implantable cardiac ICD lead during and after implantation comprising an ICD lead having a magnetic tracking sensor affixed thereto, a magnetic field generator, and a computer programmed to determine the sensor's X, Y, Z coordinates and pitch and yaw orientation in real time.

2. The system according to claim 1, wherein an absolute frame of reference is established by said external magnetic field generator.

3. The system according to claim 1, wherein said sensor on said lead is used to assist implantation of said lead.

4. The system according to claim 1, wherein a single lead with a magnetic sensor measures real time motion of an atrium outer wall of a heart to which said lead is attached.

5. The system according to claim 4, wherein absolute motion of the atrium wall is employed to monitor overall cardiac function of a patient.

6. The system according to claim 5, wherein cardiac function is monitored over time to (a) establish baseline cardiac output, (b) to monitor gradual cardiac degradation, (c) to initiate pacing, or (d) to alert medical professionals when cardiac performance falls below preset limits.

7. The system of claim 4, wherein absolute motion of the atrium is sensed, and, responsive to values outside preset limits, pacing is initiated.

8. The system of claim 4, wherein absolute motion of the atrium is sensed, and responsive to values outside preset limits, pacing is ceased.

9. The system of claim 4, wherein absolute motion of the atrium is used to optimize pacing for maximum cardiac output and/or to minimize power drain on the ICD.

10. The system according to claim 1, wherein one or more additional leads are implanted within a heart to provide multiple electrical stimulation points and/or to provide absolute positional real time information at an implantation site.

11. The system according to claim 10, wherein absolute position from multiple leads is employed to optimize voltage, current and/or lead to lead timing of electrical stimulus from an ICD to optimize cardiac output, or to minimize ICD power drain, or to interrupt uncoordinated cardiac activity and/or to resynchronize cardiac rhythm.

12. The system according to claim 1, wherein an additional magnetic tracking sensor is located inside the ICD or outside the ICD but subcutaneously above ribs to provide a patient-based frame of reference.

13. The system according to claim 12, wherein said additional sensor is employed to detect patient body motion and improve an algorithm employed to determine cardiac motion.

14. The system according to claim 12, wherein said additional sensor is employed to detect patient respiratory motion in order to improve an algorithm employed to determine cardiac motion.

15. The system according to claim 12, wherein said additional sensor is used to monitor patient respiratory motion in order to identify if a patient has ceased breathing.

16. The system according to claim 15, wherein the ICD initiates electrical stimulation responsive to sensing respiratory function outside preset parameters to reestablish respiratory function.

17. The system according to claim 16, wherein an additional electrical stimulation lead is placed in a body (outside a heart thereof) to stimulate the body to reestablish respiratory function.

18. The system according to claim 1, wherein a large inductive coupler is located within the ICD to provide electrical power from a magnetic field generated by the magnetic field generator, said electrical power being supplied to the ICD to power the ICD and/or to extend the ICD's battery life.

19. The system according to claim 1, wherein the magnetic field generator is located near a patient during periods of time when said patient is asleep.

20. The system according to claim 1, wherein the magnetic field generator is located on or inside a patient's chair, wheelchair or hospital bed.

21. The system according to claim 12, wherein the magnetic field generator is located on or inside a patient's chair, wheelchair or hospital bed.

22. The system according to claim 1, wherein the magnetic field generator is portable and can be moved with a patient.

23. The system according to claim 12, wherein the magnetic field generator is portable and can be moved with a patient.

24. The system according to claim 1, wherein an accelerometer is placed on the lead adjacent to the magnetic tracking sensor so that acceleration and displacements are simultaneously measured.
